(19) United States
(12) Plant Patent Application Publication
(10) Pub. No.: US 2016/0278272 P1
(43) Pub. Date: Sep. 22, 2016

(54) Portulaca plant named 'Dpazfchsia'

(50) ;

(71) Applicant: Gavriel Danziger, Moshav Mishmar Hashiva (IL)

(72) Inventor: Gavriel Danziger, Moshav Mishmar Hashiva (IL)

(21) Appl. No.: 14/544,979

(22) Filed: Mar. 12, 2015

(51) Int. Cl. A01H 5/00

(52) U.S. Cl. PLT/471

(57) ABSTRACT

A new and distinct Portulaca cultivar named ‘DPAZFCHSIA’ is disclosed, characterized by stable flower color, long blooming period—from Spring to Autumn, compact and semi-trailing habit. The new variety is a Portulaca, normally produced as an outdoor garden or container plant.

### LATIN NAME OF THE GENUS AND SPECIES

Portulaca oleracea

### VARIETY DENOMINATION

‘DPAZFCHSIA’

### BACKGROUND OF THE INVENTION

The new Portulaca cultivar is a product of a planned breeding program conducted by the inventor, Gavriel Danziger, in Moshav Mishmar Hashiva, Israel. The objective of the breeding program was to produce new Portulaca varieties for ornamental commercial applications. The open pollination resulting in this new variety was made during 2010.

The seed parent is the, unpatented, proprietary variety referred to as Portulaca ‘PT-9-650’. The pollen parent is unknown as it was an open pollination breeding program. The new variety was discovered in June of 2011 by the inventor in a group of seedlings resulting from the 2010 crossing, in a research greenhouse in Moshav Mishmar Hashiva, Israel.

Asexual reproduction of the new cultivar was performed by vegetative cuttings. This was first performed at a research greenhouse in Moshav Mishmar Hashiva, Israel in August of 2011 and has shown that the unique features of this cultivar are stable and reproduced true to type in more than 10 successive generations.

### SUMMARY OF THE INVENTION

The cultivar ‘DPAZFCHSIA’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DPAZFCHSIA’ These characteristics in combination distinguish ‘DPAZFCHSIA’ as a new and distinct Portulaca cultivar:
1. Compact and semi-trailing plant habit.
2. Vivid and stable flower color.
3. Long blooming period, from Spring to Autumn.

Plants of the new cultivar ‘DPAZFCHSIA’ are similar to plants of the seed parent, Portulaca ‘PT-9-650’ in most horticultural characteristics, however, plants of the new cultivar ‘DPAZFCHSIA’ differ in the following;
1. New variety has a more compact and denser habit.
2. Less trailing habit than seed parent.
3. New variety produces larger flowers.

### COMMERCIAL COMPARISON

Plants of the new cultivar ‘DPAZFCHSIA’ are comparable to the commercial variety Portulaca ‘Cupcakes Grape Jelly’. The two Portulaca varieties are similar in most horticultural characteristics; however, the new variety ‘DPAZFCHSIA’ differs in the following:
1. Leaves or the new variety are narrower in the apex and thinner overall.
2. Petal apex of the new variety is rounded while the petal apex of ‘Cupcakes Grape Jelly’ is retuse.
3. ‘DPAZFCHSIA’ has a plant habit which is semi trailing habit while the plant habit of ‘Cupcakes Grape Jelly’ is semi-upright.

Plants of the new cultivar ‘DPAZFCHSIA’ can also be comparable to the patented commercial variety Portulaca ‘SAKPOR001’ U.S. Plant Pat. No. 24,528. The two Portulaca varieties are similar in most horticultural characteristics; however, the new variety ‘DPAZFCHSIA’ differs in the following:
1. Flowers are different shade of purple; N74B while the ‘SAKPOR001’ flower color is 67A.
2. The new variety has a plant habit which is compact and semi-trailing; this comparator has a plant habit is semi-mounding and upright.
3. Plant vigor of the new variety is moderate; this comparator has a vigorous growth habit.

### BRIEF DESCRIPTION OF THE PHOTOGRAPHS

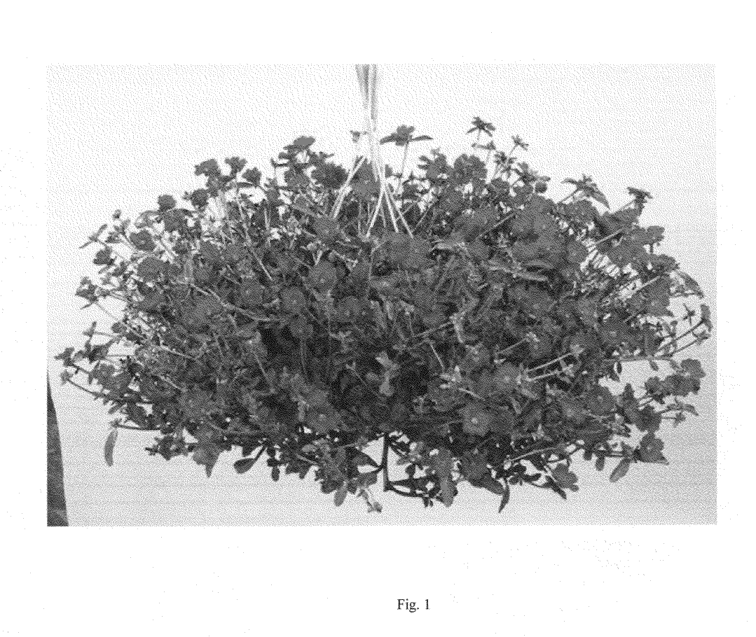

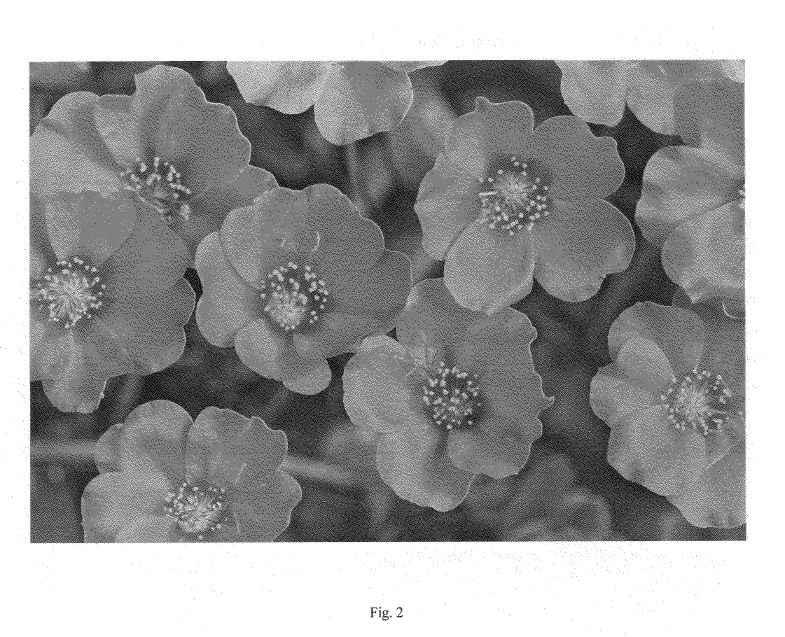

The accompanying photograph in FIG. 1 illustrates in full color a typical plant of ‘DPAZFCHSIA’ grown in a greenhouse, in a 13 cm pot. Age of the plant photographed is approximately 60 days from a rooted cutting. FIG. 2 illustrates a close up of the foliage. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Mini Colour Chart 2001 except where general terms of ordinary dictionary significance are used. The following observations and measurements describe ‘DPAZFCHSIA’ plants grown in the October in a greenhouse, in Moshav Mishmar Hashiva, Israel. The growing temperature ranged from 14° C. to 30° C. during the day and from 10° C. to 23° C. during the night. General light conditions are bright, normal sunlight. Measurements and numerical values represent averages of typical plant types.
Botanical classification.—Portulaca oleracea ‘DPAZFCHSIA’.
Propagation:
   Type of propagation typically used.—Cutting.
   Time to initiate roots.—10-14 days at approximately 18-21° C.
   Root description.—Fibrous, spreading and dense.
Plant:
   Age of plant described.—60 days from a rooted cutting.
   Container size of the plant described.—13 cm.
   Growth habit.—Semi-trailing.
   Height.—17 cm.
   Plant spread.—About 28 cm (radius).
   Growth rate.—Medium vigor, semi-trailing growth habit.
   Branching characteristics.—Tight, compact, dense branching.
   Length of primary lateral branches.—26 cm.
   Diameter of lateral branches.—0.3-0.5 cm.
   Quantity of primary lateral branches.—About 8.
   Characteristics of primary lateral branches.—Form: Horizontal. Rounded with ridges along the branch Diameter: Approximately 0.4 cm. Color: From the base RHS Greyed-orange 176A-176B. From the tip RHS Yellow-green 144A Texture: Glabrous Strength: Flexible.
   Internode length.—Approximately 3 cm.
Foliage:
   Leaf.—Arrangement: Alternate Quantity: Approximately 36 per branch. Average Length: 3.5 cm. Average Width: 1.4 cm. Shape of blade: Oblanceolate Apex: Rounded tip in mature leaves and acute tips in younger leaves. Base: Acuminate Margin: Entire Texture of top surface: Smooth Texture of bottom surface: Smooth Pubescence: Absent Aspect: Mainly flat, angle of attachment about 45° Color: Young foliage upper side: RHS Yellow-green 146A Young foliage under side: RHS Yellow-green 146B Mature foliage upper side: RHS Yellow-green 146A Mature foliage under side: RHS Yellow-green 146B Venation: Indistinguishable from leaf blade. Petiole: Absent.
Flower:
   Natural flowering season.—Spring to Autumn in Moshav, Mishmar, Israel.
   Days to flowering from rooted cutting.—About 6 to 8 weeks.
   Inflorescence and flower type and habit.—Terminal inflorescence. Single flower. Flower held horizontal.
   Rate of flower opening.—7 to 10 days from bud to fully opened flower.
   Flower longevity on plant.—Approximately 1 day.
   Persistent or self-cleaning.—Self cleaning.
   Bud.—Shape: Narrowly conical Length: Approximately 0.8-1 cm Diameter: Approximately 0.5 cm Color: Bud base: RHS Yellow-green 144A with a purplish tip near RHS Red-purple 59A.
   Flower size.—Diameter: 3.2 cm. Length: 0.5 cm.
   Corolla/petals.—Arrangement: Single flower with 5 distinct petals fused at base. Length: Approximately 1.5 cm. Width: Approximately 1.2 cm. Quantity: 5 petals Texture: Glabrous Apex: Rounded Shape: Obovate with a cuneate base. Margin: Entire Color: When opening: Upper surface: RHS Red-purple N74B Lower surface: RHS Red-purple 67C Fully opened: Upper surface: RHS Red-purple N74B Lower surface: RHS Red-purple 67C.
   Calyx/sepals.—Quantity per flower: 2 Sepals per flower Shape: Deltoid, elliptic with pointed tip Length: 0.7 cm Width: 0.5 cm Apex: Acute Base: Fused Margin: Entire Texture: Glabrous all surfaces. Color: Upper Surface: RHS Yellow-green 144B bit purplish in the tip. Lower Surface: RHS Yellow-green 144B.
   Peduncle.—None.
   Fragrance.—No.
Reproductive organs:
   Stamens.—Number: Many, around 60 Filament length: Approximately 0.2 cm.
   Anthers.—Shape: Oval, bilobed Length: Less than 0.5 mm Color: RHS Yellow-green 150D Pollen: Color: Near RHS Orange N25B Quantity: Many, around 60.
   Pistil.—Number: 1 per flower Length: 0.7 cm. Style: Length: 0.5 cm. Color: Near RHS Yellow 2B Stigma: Shape: Six branched stigma Color: Near RHS Yellow-Orange 17A and pinkish in the center Ovary Color: Near RHS Blue-Green 114B.
Other characteristics:
   Seeds and fruits.—Fruits are egg-shaped 4 mm long with a conical shape top. Seeds are dark grey with a circular shape. Minute and not possible to correctly identify R.H.S. color.
   Disease/pest resistance.—No unusual susceptibility or resistance to diseases or insects has been observed.
   Temperature tolerance.—Will not tolerate temperatures below 7° C. High temperature tolerance to 40° C.

What is claimed is:

1. A new and distinct cultivar of Portulaca plant named ‘DPAZFCHSIA’ as herein illustrated and described.
